(19) United States
(12) Patent Application Publication
(10) Pub. No.: US 2024/0374856 A1
(43) Pub. Date: Nov. 14, 2024

(54) INHALED NO FOR TREATING AN ADULT PATIENT WITH SEVERE ARDS

(71) Applicant: L'Air Liquide, Socie'te' Anonyme pour I'Etude et I'Exploitation des Proce'de's Georges Claude, Paris (FR)

(72) Inventor: Laurent LECOURT, Cachan (FR)

(21) Appl. No.: 18/659,688

(22) Filed: May 9, 2024

(30) Foreign Application Priority Data
May 10, 2023 (FR) 2304626

(51) Int. Cl. A61M 16/12; A61M 16/04

(52) U.S. Cl. CPC A61M 16/12; A61M 16/04; A61M 2202/0275

(57) ABSTRACT

The invention relates to a gaseous drug containing nitric oxide (NO) gas, such as an NO/nitrogen mixture, for use in the treatment of a patient suffering from acute respiratory distress syndrome (ARDS), comprising administration by inhalation of said gaseous drug to said patient. The patient is an adult suffering from severe ARDS characterized by a severity index (PaO₂/FiO₂) of less than 80 mmHg, a partial pressure of carbon dioxide (PaCO₂) of at least 60 mm Hg and a blood pH of less than 7.25. Preferably, the gaseous drug contains from 200 to 2000 ppmv of NO, with the remainder being nitrogen.

### CROSS REFERENCE TO RELATED APPLICATIONS

This application claims the benefit of priority under 35 U.S.C. § 119 (a) and (b) to French Patent Application No. 2304626, filed May 10, 2023, the entire contents of which are incorporated herein by reference.

### BACKGROUND

The invention relates to a gaseous drug containing nitric oxide (NO), in particular an NO/nitrogen mixture, which can be administered by inhalation to treat an adult individual, i.e. an adult patient, suffering from severe acute respiratory distress syndrome (ARDS), more specifically a subpopulation of severe ARDS patients who are still hypoxaemic, despite appropriate ventilatory management.

Patients with acute respiratory distress syndrome or ARDS represent about 10% of patients hospitalized in intensive care units. Prior to the Covid-19 pandemic, the incidence rate of ARDS in the general adult population ranged from 5 to 86 per 100 000. ARDS-related mortality in this adult population was high, ranging from 24% to 60% depending on the age of the patients and their general health. ARDS is therefore a serious public health problem.

ARDS is characterized by severe respiratory failure caused by acute diffuse inflammatory lung disease leading to increased permeability of the alveolar capillaries and pulmonary oedema referred to as acute lung injury. In other words, it causes a pulmonary inflammatory process that leads to pulmonary oedema rich in non-hydrostatic proteins, and to negative health consequences for patients suffering from it, namely pronounced hypoxaemia, a drop in pulmonary compliance and an increase in intrapulmonary shunts and dead spaces.

Strictly speaking, ARDS is not a disease but a syndrome illustrated by numerous clinical, paraclinical and physiological criteria.

According to a recent definition, known as the “Berlin Definition”, from a working group of the European Society of Intensive Care (ESICM), the diagnosis of ARDS is made if a patient displays, for 7 consecutive days, a known clinical condition or new or worsening respiratory symptoms, a combination of acute hypoxaemia resulting in a PaO₂/FiO₂ severity index <300 mmHg, for an artificially ventilated patient with positive end-expiratory pressure (PEEP) of at least 5 cmH₂O, and bilateral opacities that are not fully explained by left heart failure or fluid overload.

Also under the “Berlin Definition”, the PaO₂/FiO₂ severity index is used to distinguish ARDS severities, namely:
mild ARDS: 200 mmHg<PaO₂/FiO₂≤300 mmHg,
moderate ARDS: 100 mmHg<PaO₂/FiO₂≤200 mmHg,
severe ARDS: PaO₂/FiO₂≤100 mmHg.

Currently, in an attempt to treat ARDS, the main therapeutic strategy is to ensure adequate gas exchange while minimizing the risk of ventilator-induced lung injury (VILI) caused by the medical ventilator used to ventilate the patient.

In other words, in order to treat patients with ARDS, the latter are usually mechanically ventilated and, if ventilation is insufficient or ineffective, the treatment can be supplemented by additional non-pharmacological therapeutic actions such as ventilation “in ventral decubitus”, the patient being placed on the stomach for a few hours a day, or extracorporeal circulation (ECC), or pharmacological actions.

Thus, as an adjuvant pharmacological therapeutic action, the use of pulmonary vasodilator drugs with in particular inhaled nitric oxide (iON), such as an NO/nitrogen mixture, has already been proposed for treating patients in respiratory failure who are suffering from ARDS, whatever the origin, i.e. infection, traumatic or something else, such as patients infected with a coronavirus, such as Covid-19, or ARDS of another aetiology.

Until now, the efficacy of iNO in the treatment of ARDS has been limited to an improvement in arterial oxygenation and/or pulmonary arterial pressure, but without any impact on the outcome for the patient in intensive care, in particular in terms of mortality/survival.

Therefore and following the general review of iNO studies in ARDS, reported by F. Gebistorf et al, Inhaled nitric oxide for acute respiratory distress syndrome (ARDS) in children and adults; Cochrane Database of Systematic Reviews 2016, Issue 6. Art. No.: CD002787), iNO is generally not recommended, or is even advised against, in ARDS management recommendations.

Consequently, no marketing authorization (MA) for iNO includes such a therapeutic indication, as evidenced by the Summary of Product Characteristics (SPC) of the NO-based gaseous drug called Inomax® from INO Therapeutics®, dated 1999, paragraph 14.2 of which on the use of NO in ARDS is entitled: Ineffective in Adult Respiratory Distress Syndrome (ARDS). In other words, inhaled NO is also not recommended for the treatment of ARDS in adults because it is considered to be ineffective.

Also known, moreover, is WO2012/061264 which proposes the use of NO to treat ARDS with administration of NO by inhalation at a low dose, i.e. 0.01 to 100 ppm, for a period of 28 days to 7 months to improve lung function in patients with a PaO₂/FiO₂ severity index which can be up to 250. However, the patients tested, namely children, have a moderate severity index, according to the “Berlin definition”, since their PaO₂/FiO₂ index is 140.5+/−43.4. No improvement in mortality and short-term effects (i.e. 6 months) in the patients was observed. In the longer term, the results are not significant. Moreover, the document stresses that additional “long-term” studies will be necessary. It is understood that this document is limited to a paediatric population affected by a rather moderate ARDS and shows effects on oxygenation but no effect on improving patient survival (i.e. mortality).

Similarly, WO 2015/106115 proposes the use of inhaled NO to treat ARDS in children less than 16 years of age with administration of NO at a very low dose, i.e. less than 10 ppm, for 2 days to 2 months. Again, the patients tested have a moderate PaO₂/FiO₂ severity index of 140.5+/−43.4. Once again, no improvement in the short-term mortality of patients is observed.

Mention may also be made of FR 2990858 and WO 2013/175087, which propose the use of inhaled NO to treat various lung diseases, including ARDS. These documents are very general and do not focus on any particular patient population. They contribute nothing more than the previous documents.

Other existing scientific papers themselves also conclude that there has been no significant improvement in mortality in ARDS patients treated by NO inhalation (i.e. iNO). It is even generally recommended not to use iNO in these patients because of the absence of evidence of any improvement in their mortality compared to the risk of renal disorders that may result from the use of iNO. In this respect, mention may be made of:
Intensive Care Medicine, Springer-Verlag 2004®, Consensus Conference, Inhaled Nitric Oxide therapy in neonates and children: reaching a European consensus, D. J. MacRae et al, published 13 Jan. 2004, para.: Use of inhaled nitric oxide in paediatric acute lung injury and acute respiratory distress syndrome, which emphasizes a non-efficacy of iNO on mortality and only a transient improvement regarding oxygenation;
Pilbeam, Mechanical ventilation, 4^{th} Edition, Special Techniques in Mechanical Ventilation, Section IV: Nitric Oxide, 2006® Mosby Inc., para. ARDS, which reports that results from 4 large studies showed no benefit of iNO on patient mortality despite an improvement in oxygenation.

In fact, most of these publications indicate that the patients tested suffered from ARDS of varying severity according to the Berlin classification, usually moderate or mild, and that they had not received adequate assisted ventilation prior to being placed on iNO, typically protective ventilation with the patient placed on the stomach in order to limit the pressure exerted on the lungs of said patient and to improve gas exchange.

In general, all the studies carried out to date to evaluate the effects of inhaled NO in the treatment of ARDS have shown, at best, a transient improvement in arterial oxygenation but this is never correlated with an improvement in survival (i.e. mortality rate) of the patients tested.

Also known is the publication: Syndrome de Détresse Respiratoire Aigüe [Acute Respiratory Distress Syndrome], by J-C. Chevrolet et al., EMC-Pneumologie 1 (2004), 143-186, which addresses an overview on ARDS, speaks in particular of the severity index and lists various treatment methods that can be envisaged as treatment. Inhaled NO is one of them. However, this document classifies NO as a routine treatment not recommended for ARDS (see Table 4 on page 170) and indicates that caution should be exercised with respect to such use of NO, while specifying that no study this has demonstrated any effect of NO on patient survival (page 181). It concludes that, despite all the advances in the treatment of ARDS, there is no current therapy that can be used at the patient bedside to treat ARDS. Therefore, this document does not recommend the use of NO but, on the contrary, discourages its use since it is presented as having no effect improving patient survival.

One problem is therefore that of being able to improve the arterial oxygenation of one (or more) patient but also their survival rate, that is to say to reduce mortality, in particular for an adult patient suffering from severe ARDS presenting criteria of severity, namely those included in the criteria for placing under ECMO according to the “EOLIA” study, referenced: A. Combes et al.; Extracorporeal Membrane Oxygenation for Severe Acute Respiratory Distress Syndrome; New Engl. J. Med.; 2018; 378:1965-75.

A solution according to the invention relates to a gaseous drug containing nitrogen monoxide (NO) gas for use in treating an adult patient suffering from acute respiratory distress syndrome (ARDS) comprising administration by inhalation of said gaseous drug to said patient.

According to the invention, the patient is an adult suffering from severe ARDS characterized by:
(i) a severity index such that: PaO₂/FiO₂ $80 mmHg and/or
(ii) a carbon dioxide partial pressure (PaCO₂) and a blood pH such that: PaCO₂≥60 mm Hg and pH<7.25,
   where: PaO₂, PaCO₂ and the pH are measured in the patient prior to the administration of the gaseous drug.

In the context of the invention, it is considered that the patient's state of health has improved due to the administration of iNO, if it is found that said patient no longer meets the criteria for placing on ECMO, namely:
an improvement in the severity index of said patient, i.e. PaO₂/FiO₂>80 mmHg or
a PaCO₂<60 mmHg and a pH≥7.25.

In order to determine the severity index of a patient, the values of PaO₂, PaCO₂ and pH are measured, determined or evaluated in the patient in question, i.e. the patient to be treated, before any administration of the gaseous drug, i.e. of the gaseous drug containing NO gas according to the invention.

As indicated below, the PaCO₂ and PaO₂ are determined by arterial blood gas or “blood gases”, typically by taking a blood sample prior to any administration of the NO-based gaseous drug, i.e. prior to treatment with iNO. Such parameters are conventionally recorded, in particular after placing the patient under oxygen (i.e. administration of a given FiO₂), for example during care of said patient.

In general, blood gases allow monitoring of the patient's gas exchange by providing the PaO₂ and PaCO₂ values and the saturations. The values can be taken several times a day in an intensive care unit, in particular depending on the severity of the patient's condition. The blood gas record also mentions FiO₂ since PaO₂ and saturation values in particular correlate with the proportion of oxygen delivered to the patient, i.e. FiO₂.

The fraction of inspired oxygen or FiO₂ is the amount of oxygen (vol. %) present in the gas mixture inhaled by the patient, i.e. the amount of oxygen supplied to the patient. It corresponds to the O₂ concentration set by the physician and delivered by the medical ventilator. The FiO₂ can reach 100% O₂ for a highly affected patient, i.e. can correspond to a flow of pure oxygen. Typically, the FiO₂ in ARDS patients is between 50 and 100 vol. % oxygen, depending on the severity of the ARDS.

In other words, in the context of the invention, only patients with severe ARDS exhibiting the following blood gas parameters: a severity index (PaO₂/FiO₂ ratio) of less than or equal to 80 mmHg or a carbon dioxide partial pressure (PaCO₂) such that: PaCO₂>60 mmHg and a blood pH such that: pH<7.25 where PaCO₂ and PH are measured in the patient prior to administration of the gaseous drug containing NO gas according to the invention, are involved since these blood gas parameters correspond to criteria for placing under ECMO (EOLIA study) which is generally the last resort after failure of conventional ventilation (ECMO for extracorporeal membrane oxygenation).

ECMO is a technique for direct oxygenation of blood by placing the patient on extracorporeal circulation (ECC) using a pump to generate extracorporeal blood flow and an oxygenation membrane(s) to schematically rid the blood of CO₂ contained therein and enrich the blood with oxygen.

Indeed, it has been demonstrated in the context of the present invention that, contrary to what is usually accepted in the prior art, namely that NO has no significant effect on the survival/mortality of patients suffering from ARDS, administration of iNO, surprisingly, significantly improves survival in a particular subpopulation of adult patients, namely the patients most severely affected with ARDS, i.e. severe ARDS, of various aetiologies, for example patients with viral or bacterial pneumonia, notably those infected with a virus, such as a coronavirus, for example Covid-19, or other.

Indeed, until the present invention, no study had demonstrated an efficacy of inhaled NO on the survival rate of adult patients suffering from severe ARDS, regardless of the ARDS patient populations studied.

In the context of the invention:
PaO₂: is the partial pressure of oxygen (O₂) and reflects the amount of oxygen in an individual's blood. It is determined by arterial blood gas (i.e. blood test). It is expressed here in mmHg.
FiO₂: is the inspired fraction of oxygen, is the amount or proportion of oxygen present in the gas administered by inhalation to an individual and is expressed in vol. %. It is set on the medical ventilator by the caregiver. For ARDS patients, it is generally at least 40 vol. %, or even at least 50 vol. % and can reach about 100 vol. %, which corresponds to (almost) pure oxygen.
PaCO₂ is the partial pressure of carbon dioxide (CO₂) and reflects the amount or proportion of CO₂ in an individual's arterial blood. It is determined by arterial blood gas. It is expressed here in mmHg.
the PaO₂/FiO₂ severity index reflects the degree of oxygenation of a patient under artificial or assisted ventilation, i.e. the ratio of arterial oxygenation (i.e. the amount of oxygen in the blood) as a function of the amount of oxygen delivered by the assisted ventilation, i.e. by the medical ventilator. By way of indication, a “normal” value is about 450 mmHg. It is expressed here in mmHg. This ratio, as seen above, is an indicator for defining the severity stages of ARDS according to the Berlin definition.
the pH reflects the degree of acidity or alkalinity of the blood. As an indication, a normal pH is between 7.35 and 7.45.
ppmv: means parts per million in volume.
vol. %: means percentage by volume.
air: it is considered to contain approximately 20 to 21 vol. % oxygen (O₂) and approximately 78% nitrogen (N₂). The other species present (i.e. argon, CO₂, helium, etc.) are considered as unavoidable impurities having no influence on the efficacy of the iNO treatment; they are therefore ignored.

Depending on the embodiment under consideration, the gaseous drug used according to the invention may comprise one or more of the following features:
it is formed of a mixture of NO and nitrogen (N₂);
it is formed of a mixture of NO and nitrogen containing from 200 to 2000 ppmv of NO, preferably between 200 and 1500 ppmv;
it is formed of a mixture of NO and nitrogen containing from 200 to 1000 ppmv, preferably between 400 and 900 ppmv of NO, the remainder being nitrogen;
advantageously, it is formed of a mixture of NO and nitrogen containing 800 ppmv of NO, the remainder being nitrogen;
alternatively, it is formed of a mixture of NO and nitrogen containing 450 ppmv of NO, the remainder being nitrogen;
alternatively, it is formed of a mixture of NO and nitrogen containing 225 ppmv of NO, the remainder being nitrogen;
it is diluted (before inhalation by the patient) with an oxygen-containing respiratory gas, i.e. before administration to the patient;
it is diluted (before inhalation by the patient) with a respiratory gas containing at least approximately 20 vol. % of oxygen, preferably at least approximately 21 vol. % of oxygen, i.e. before administration to the patient, preferably at least 50% O₂;
it is diluted (before inhalation by the patient) with an oxygen-containing respiratory gas, before administration to the patient, for example a flow of pure oxygen or an O₂/N₂ mixture;
it is diluted with a respiratory gas to obtain a final mixture (i.e. gas inhaled by the patient) containing oxygen, nitrogen and less than 40 ppmv of NO as the dose of NO to be administered, i.e. as dosage;
preferably, the dose of NO to be administered (in the final mixture after dilution) is between 1 and 30 ppmv, more preferably from 1 to 20 ppmv of NO, advantageously about 10 ppmv;
the oxygen-containing respiratory gas is supplied by a medical ventilator;
the oxygen-containing respiratory gas is an O₂/N₂ mixture or pure oxygen;
the patient is an adult at least 18 years old, preferably at least 30 years old, preferably at least 40 years old, typically 45 to 95 years old, preferably between 50 and 90 years old;
it is administered by inhalation for several hours, preferably at least 24 hours, typically between 24 hours and 96 hours, or even more in the most serious cases;
it is administered by inhalation using a tracheal intubation tube (i.e. the patient is intubated);
it is packaged in (at least) a gas container, typically a gas cylinder;
alternatively, it is produced on site (i.e. extemporaneously) by means of an NO-generating device, such as for example that described by EP 3509684;
the gas container(s), typically a gas cylinder, contain(s) an NO/N₂ mixture consisting of 450 ppmv or 800 ppmv of NO and nitrogen for the remainder (and possibly unavoidable impurities);
the patient is under artificial ventilation and intubated, that is to say under invasive ventilation, typically in intensive care;
the patient to be treated needs treatment by NO inhalation;
the patient is bedridden, i.e. lying in bed, during inhalation of iNO;
the patient is a man or a woman, or other;
the patient presents (before treatment) refractory hypoxaemia despite assisted ventilation and possibly a ventral position (i.e. ventral decubitus), that is to say that refractory hypoxaemia is not treatable by assisted ventilation and prone position;
the patient has one or more comorbidities selected from systemic hypertension, diabetes, immunodeficiency and chronic obstructive pulmonary disease (COPD);
NO is the active ingredient of the gaseous drug;
the gas container(s), typically a gas cylinder, contains the NO/N₂ mixture compressed to a pressure of at least 150 bar abs, typically about 200 bar abs, measured when the container is full, i.e. before any use of the gas;
the patient has severe ARDS characterized by a severity index such that: 35 mmHg≤PaO₂/FiO₂≤80 mmHg;
the patient has severe ARDS characterized by a severity index such that: 40 mmHg≤PaO₂/FiO₂≤80 mmHg;
the patient has severe ARDS characterized by a severity index such that: 50 mmHg≤PaO₂/FiO₂≤80 mmHg;
the patient has severe ARDS characterized by a severity index such that: 60 mmHg≤PaO₂/FiO₂≤80 mmHg.

The invention also relates to ventilation equipment for providing ventilatory assistance to an adult patient with severe ARDS, comprising:
at least one gas cylinder, i.e. compressed gas container, containing a gaseous drug according to the invention formed from an NO/N₂ mixture,
a medical ventilator to supply a flow of a respiratory gas containing oxygen (i.e. at least approximately 21 vol. %), to a patient circuit, and
a gas supply apparatus supplied with a NO/N₂ mixture by said at least one gas cylinder, and fluidly connected to said patient circuit for injecting the NO/N₂ mixture into the oxygen-containing respiratory gas flow circulating in the patient circuit, that is to say for mixing the NO/N₂ flow and the oxygen-containing respiratory gas flow within said patient circuit.

Depending on the embodiment in question, the ventilation equipment of the invention may comprise one or more of the following characteristics:
the gas cylinder contains an NO/N₂ mixture containing from 100 to 2000 ppmv of NO and nitrogen for the remainder, preferably from 100 to 1500 ppmv, preferably from 100 to 1000 ppmv, typically 450 or 800 ppmv of NO;
the oxygen-containing respiratory gas flow is an O₂/N₂ mixture (e.g. at least 40 vol. %, or even at least 50 vol. %) or pure oxygen;
the patient circuit conveys a final gas flow containing a desired proportion or dose of NO of between 1 and 40 ppmv, preferably less than approximately 20 ppmv of NO;
the patient circuit conveys a final gas flow containing a desired proportion or dose of NO of about 10 ppmv;
the final gas flow containing the desired NO dose is obtained by injecting the NO/N₂ mixture originating from the gas delivery apparatus into the oxygen-containing respiratory gas flow originating from the medical ventilator;
the patient circuit supplies a tracheal intubation tube with the final gas flow containing the desired NO dose;
the patient circuit comprises an inhalation branch and an exhalation branch;
the inhalation branch and the exhalation branch are fluidly connected to a connecting or joining piece, such as a Y-piece;
the tracheal intubation tube is fluidly connected to the connecting piece, i.e. the Y-piece;
the inhalation branch is fluidly connected, upstream, to the medical ventilator and, downstream, to the tracheal intubation tube, via the connecting piece, i.e. the Y-piece, so as to convey in particular the final gas flow containing NO at the desired dose;
the exhalation branch is fluidly connected, upstream, to the tracheal intubation tube via the connecting piece, i.e. the Y-piece, and, downstream, to the medical ventilator so as to convey the gas leaving the patient's lungs (i.e. CO₂-rich gas) to the medical ventilator;
the inhalation branch comprises a gas humidifier for adding water vapour to the final gas flow containing NO at the desired dose;
the inhalation branch further comprises a flow sensor electrically connected to the gas supply apparatus so as to provide it with flow measurements for the oxygen-containing respiratory gas flow originating from the medical ventilator;
the gas delivery apparatus comprises a graphical user interface (GUI) comprising a graphical display;
the graphical display is configured to display one or more virtual or analogue selection keys that can be actuated by the user, in particular by means of a finger press;
the graphical display includes a touch panel;
the graphical display is a colour display;
the GUI is configured to allow the user to set or select a desired NO dose, in particular via one or more virtual selection keys displayed on the graphical display;
the gas supply apparatus comprises control means configured to process the flow rate measurements coming from the flow sensor and to control the delivery of the NO/N₂ mixture flow in response to the processing of the flow rate measurements carried out and as a function of the desired NO dose set or selected by the user;
the gas supply apparatus and the medical ventilator are supplied with electric power, notably with mains power (110/220 V) and/or one more internal rechargeable batteries.

Such ventilation equipment is used to treat an adult patient suffering from severe ARDS as described above, said treatment comprising the administration by inhalation, to said patient, of a desired dose of NO of less than 40 ppmv, typically less than 20 ppmv, via a tracheal intubation tube.

The invention further relates to a method of treating an adult patient, i.e. an adult individual, suffering from severe acute respiratory distress syndrome (ARDS) characterized by a severity index such that: PaO₂/FiO₂≤80 mmHg, or a partial pressure of carbon dioxide (PaCO₂) and a blood pH such that: PaCO₂≥60 mm Hg and pH<7.25, where: PaO₂, PaCO₂ and PH are measured in the patient prior to administration of the gaseous drug, in which a gaseous drug containing nitric oxide (NO) gas according to the invention, i.e. an NO/N₂ gas mixture, is administered by inhalation to the patient to be treated in order to improve the patient's blood oxygenation and to increase the latter's chances of survival (i.e. decrease the latter's risk of death).

Depending on the embodiment under consideration, the treatment method of the invention can comprise one or more of the following features:
the NO-containing gaseous drug, typically a NO/N₂ gaseous mixture, is diluted with an oxygen-containing respiratory gas containing at least 20 vol. %) oxygen, prior to administration to the patient;
a patient with severe acute respiratory distress syndrome (ARDS) is identified, characterized by a severity index or a partial pressure of carbon dioxide (PaCO₂) and a blood pH as defined above;
the identification of the ARDS patient comprises performing an arterial blood gas or “blood gases” test in the patient, in particular to determine PaCO₂ and PaO₂;
the patient presents (before treatment) refractory hypoxaemia despite assisted ventilation and/or prone positioning (i.e. ventral decubitus);
the patient is under invasive artificial ventilation and intubated, typically is hospitalized in an intensive care unit of a hospital or the like;
the patient has one or more comorbidities selected from hypertension, diabetes, immunodeficiency and chronic obstructive pulmonary disease (COPD);
the patient to be treated needs treatment by NO inhalation;
the patient is bedridden, i.e. lying in bed, during inhalation of iNO;
the patient is a man or a woman, or other;
the patient is an adult at least 18 years old, preferably at least 30 years old, preferably at least 40 years old, typically 45 to 90 years old, preferably between 50 and 80 years old;
the gaseous drug is administered by inhalation using a tracheal intubation tube (i.e. the patient is intubated);
the gaseous drug is administered insertion of the tracheal intubation tube (i.e. preferably, quickly after intubation of the patient);
the gaseous drug is formed of a mixture of NO and nitrogen (N₂), that is to say an NO/N₂ gas mixture;
the mixture consisting of NO and nitrogen contains from 200 to 2000 ppmv of NO, preferably from 200 to 1000 ppmv of NO, more preferably from 400 to 900 ppmv of NO, the remainder being nitrogen;
advantageously, the mixture of NO and nitrogen contains 800 ppmv of NO, the remainder being nitrogen;
alternatively, the mixture of NO and nitrogen contains 225 ppmv of NO, the remainder being nitrogen;
alternatively again, the mixture of NO and nitrogen contains 450 ppmv of NO, the remainder being nitrogen;
the NO/N₂ gaseous drug is diluted with a respiratory gas containing (at least 20 vol. %) oxygen, prior to administration to the patient, so as to obtain a final mixture containing oxygen, nitrogen and less than 40 ppmv of NO as a dose of NO to be administered, i.e. as a dosage, preferably at least about 21 vol. % oxygen;
preferably, the dose of NO to be administered to the patient is between 1 and 30 ppmv, more preferably from 1 to 20 ppmv of NO, advantageously about 10 ppmv;
the oxygen-containing respiratory gas is supplied by a medical ventilator, it is preferably an O₂/N₂ mixture or pure O₂;
the gaseous drug is administered by inhalation for several hours, preferably at least 24 hours, typically between 24 hours and 96 hours, or even more in the most serious cases;
the administration of iNO is stopped gradually (at the end of the treatment) by reducing the dose of NO (for example 10 ppmv) by 1 ppmv every 30 min to 60 min;
the administration of iNO is stopped gradually by monitoring the oxygenation of the patient, that is to say according to a withdrawal protocol comprising a gradual reduction of the dose of NO administered in order to avoid the risks of rebound effect associated with a sudden cessation of an administration of inhaled NO;
the administration of iNO is stopped after a given duration of treatment with iNO, for example a maximum duration of about several days or less;
alternatively, the administration of iNO is stopped when the patient's state of health has improved, in particular after improvement of gas exchange and a return to correct arterial oxygenation

The invention will now be better understood by virtue of the following detailed description, given by way of illustration but not limiting, reporting the results obtained during comparative trials carried out in patients suffering from ARDS and with reference to the appended figure in which FIG. 1 represents an embodiment of ventilation equipment making it possible to supply iNO to a patient.

A study was carried out in order to show the efficacy of the inhalable gaseous NO-based drug according to the invention. In the context of this study, 300 individuals, i.e. men and women, called “patients”, who had ARDS and were treated with iNO were selected in several hospitals. These ARDS patients were classified according to their severity by assessing their oxygenation parameters, namely PaCO₂, PaO₂ and blood pH, prior to any administration of iNO. Of this population, a subpopulation of the most serious patients was defined including those with criteria for recourse to ECMO.

Thus, of these 300 patients, only a subpopulation of 62 patients exhibits these criteria for ECMO, called “ECMO patients”, namely a PaO₂/FiO₂ ratio or severity index such that: PaO₂/FiO₂≤80 mmHg, or having a partial pressure of carbon dioxide (PaCO₂) such that: PaCO₂≥60 mm Hg and a blood pH such that pH<7.25. This subpopulation of 62 “ECMO patients” included 49 men and 13 women 53 to 71 years old, with an average age of 66.

These patients are intubated and ventilated by a medical ventilator. To do this, ventilation equipment such as that described in FIG. 1 and detailed below was used. It should be specified that these patients are not placed on ECMO (extracorporeal membrane oxygenation), that is to say on extracorporeal blood circulation, although they meet the criteria for placing on ECMO, but are nevertheless kept on artificial ventilation.

iNO was administered to them by inhalation, via a tracheal intubation tube, in the form of a mixture of NO and nitrogen (the remainder) which is mixed with a non-hypoxic oxygen gas in an appropriate amount, namely in this case containing at least 60% oxygen (the remainder nitrogen), typically pure oxygen, which corresponds to an FiO₂ equal to 100%.

After dilution of the gases (i.e. NO/N₂ and in this case O₂), the final NO/O₂/N₂ mixture obtained is administered by inhalation to the patients tested who have been intubated. This final mixture contains about 10 ppmv of NO (desired average value), with the remainder being nitrogen and oxygen. However, it should be noted that isolated fluctuations were observed during the administration of iNO, leading to administered NO content values of between about 6 and 13 ppmv. The inhalation of the NO gas is carried out continuously for a predetermined period of several hours.

Thus, the results obtained within 6 hours following the inhalation of iNO are given in Table 1. Although the response obtained for each patient has been evaluated at various times, after the start of the iNO administration, the most clinically beneficial thing is to evaluate the response at least 6 hours, i.e. at least 6 hours after the start of the iNO inhalation, because this early response makes it possible to visualize a specific effect of the administered NO.

The response to NO is considered to be a loss of the criteria for placing on ECMO after administration of the iNO, within 6 hours of the start of inhalation of the NO-based gas.

Table 1 therefore compares the patients referred to as “responders” (i.e. loss of ECMO criteria within 6 hours following placing on iNO) with the patients referred to as “non-responders” (i.e. persistence of ECMO criteria after administration of iNO).

TABLE 1

| | “Non-responder” | “Responder” |
|---|---|---|
| | patients | patients who |
| | who still meet | no longer meet |
| | the ECMO | the ECMO |
| Patients tested | criteria | criteria |
| 62 patients | 32 | 30 |
| % | 51.6% | 48.4% |
| Men | 24 | 25 |
| Women | 8 | 5 |
| Average age (years) | 65 | 66 |
| Average Weight (kg) | 89 | 86 |
| Average BMI (kg/m²) | 29 | 29 |

BMI: Body mass index

As can be seen, among the 62 patients tested, 30 patients “responded” positively to the administration of iNO, that is to say that the state of their health improved, i.e. 48.4% of the patients tested.

The patients were considered to be “responders” to inhaled nitric oxide (iNO) when the ECMO criteria, as “positive response” criteria, were seen to disappear in said patients within 6 hours of inhaling iNO, resulting in them showing improved oxygenation.

Conversely, the “non-responder” patients are those for whom such an improvement is not observed and who retain criteria for placing on ECMO.

These initial results show that iNO can act with efficacy on a majority of patients suffering from ARDS and presenting at the start, i.e. during the diagnosis of their condition, the criteria for placing on ECMO. In other words, these results confirm the advantage of NO in improving oxygenation parameters and, in this case, in a population of the most serious patients.

It should be noted that the demographic characteristics of this subpopulation are on the whole similar to the overall population, for example some have comorbidities, for example such as hypertension, diabetes, immunodeficiency or COPD.

Table 2 gives the distribution of these 62 patients tested in terms of their possible prone positioning between the time of their intubation and the start of iNO administration.

TABLE 2

| | “Non-responder” patients | “Responder” patients who |
|---|---|---|
| Prone | who still meet the | no longer meet the |
| positioning | ECMO criteria | ECMO criteria |
| yes | 20 (62.5%) | 17 (56.7%) |
| No | 12 (37.5%) | 13 (43.3%) |

It is noted that prone positioning does not seem to be a determining criterion in the responder patients, whereas it is more significant in the non-responder patients. However, once again, it should be recalled that the demographic characteristics of this subpopulation are on the whole similar to the overall population.

Moreover, Table 3 gives the progression of the average values of the PaO₂/FiO₂ ratio and of the partial pressure of oxygen PaO₂ in the 62 patients tested, measured before and after administration of iNO (i.e. after 6 h).

TABLE 3

| | “Non-responder” | “Responder” |
|---|---|---|
| | patients | patients who |
| | who still meet | no longer meet |
| | the ECMO | the ECMO |
| In mmHg | criteria N = 32 | criteria N = 30 |
| Average value of the | 68.3 (17.0) | 71.4 (27.2) |
| PaO₂/FiO₂ ratio before | | |
| administration of iNO | | |
| Average value of the | 68.1 (23.6) | 121.0 (38.5) |
| PaO₂/FiO₂ ratio after | | |
| administration of iNO | | |
| Progression of the | −0.3% | 69.4% |
| PaO₂/FiO₂ ratio before | | |
| and after NO | | |
| PaO₂ value before | 65 | 68 |
| administration of iNO | | |
| PaO₂ value after | 64 | 104 |
| administration of iNO | | |
| Progression of PaO₂ | −1.5% | 52.9% |
| before and after NO. | | |
| Average PaCO₂ value | 56.7 (12.9) | 49.4 (12.0) |
| before administration of | | |
| iNO | | |
| Average PaCO₂ value | 57.3 (11.0) | 47.6 (8.7) |
| after administration of | | |
| INO | | |

It is found that the administration of inhaled NO results in a significant improvement (approx. +53% for PaO₂/FiO₂ ratio and PaO₂) of the amount of oxygen in the blood of the “responder” patients, i.e. their blood oxygenation, whereas it has no influence in the “non-responder” patients. This demonstrates that iNO notably promotes the passage of oxygen from the lungs to the blood of “responder” patients with severe ARDS.

In addition, Table 4 records the results relating to the evaluated mortality of the patients tested, after 6 hours of administration of iNO, this mortality being judged at 28 days and over the total duration of hospitalization.

TABLE 4

| | “Non-responder” | “Responder” |
|---|---|---|
| | patients who | patients who |
| | still meet | no longer meet |
| Death | the ECMO | the ECMO |
| Number (%) | criteria N = 32 | criteria N = 30 |
| Hospital mortality rate | 84.4% | 60% |
| Mortality rate at 28 days | 68.8% | 50.7% |

The tests carried out show that the particularly notable effect of iNO administration on the survival rate of the 30 “responder” patients with severe ARDS (after 6 hours of NO inhalation) since 40% of these patients survived, whereas in the group of 32 “non-responder” patients, only 15.6% of patients survived in the end.

In other words, mortality decreased significantly in the group of very severe ARDS patients with criteria for placing on ECMO, who responded positively after iNO, i.e. they no longer met the ECMO criteria within 6 hours of iNO administration.

More generally, among the 300 patients tested at the start, the “in-hospital” mortality was 65% in the total population and 72% in the subpopulation of severe ARDS patients (according to the Berlin definition).

However, among the “responder” patients who initially met ECMO criteria (i.e. 62 “ECMO” patients), mortality was only 60%, i.e. significantly lower than that of the “non-responder” patients, for whom mortality was 84.4% (p=0.032), and also lower than that of the overall population tested (300 patients) which is 65%.

This comparison with the overall population confirms the effect of iNO, which acts with efficacy on oxygenation but also, surprisingly, on the mortality of the subpopulation in question.

Moreover, when considering the overall population of patients tested, there is no change in survival in this general population but only in the oxygenation of the patients, thereby confirming previous knowledge of NO in the treatment of ARDS, which concludes that iNO is ineffective in ARDS, particularly with respect to any improvement in patient mortality.

The trials carried out in the context of the present invention therefore demonstrate the efficacy of iNO in the treatment of a particular subpopulation of patients suffering from severe ARDS characterized by a severity index such that: PaO₂/FiO₂≤80 mmHg or a partial pressure of carbon dioxide (PaCO₂) and a blood pH such that: PaCO₂≥60 mm Hg and pH<7.25, where: PaO₂, PaCO₂ and/or PH are measured in the patient prior to administration of the gaseous drug and FiO₂ is the inhaled fraction of oxygen, i.e. the amount or proportion of oxygen present in the gas administered by inhalation to the patient.

Advantageously, the administration of iNO is as soon as possible after intubation of the patient, that is to say that NO given more rapidly after intubation seems to be more efficacious in this subpopulation of patients. Administration of NO rapidly and preferably within 24 hours after intubation is therefore recommended.

In general, the results obtained are particularly surprising given that iNO is reputed, according to the prior art, not to be efficacious in terms of improving the survival/mortality of patients suffering from ARDS.

However, the trials carried out show that iNO improves not only the oxygenation but also the survival (i.e. reduction in mortality) of a particular subpopulation of patients suffering from ARDS, namely those patients suffering from severe ARDS and presenting, at the outset (before administration of iNO), criteria for placing under ECMO as explained above. Administration of iNO to these patients makes it possible to improve not only their state of health, i.e. oxygenation, but also their survival, which has never been demonstrated hitherto.

### BRIEF DESCRIPTION OF THE DRAWING

For a further understanding of the nature and objects for the present invention, reference should be made to the following detailed description, taken in conjunction with the accompanying drawings, in which like elements are given the same or analogous reference numbers and wherein:

FIG. 1 illustrates an exemplary medical nitric oxide gas delivery device used in the methods described and claimed herein.

### DETAILED DESCRIPTION OF PREFERRED EMBODIMENTS

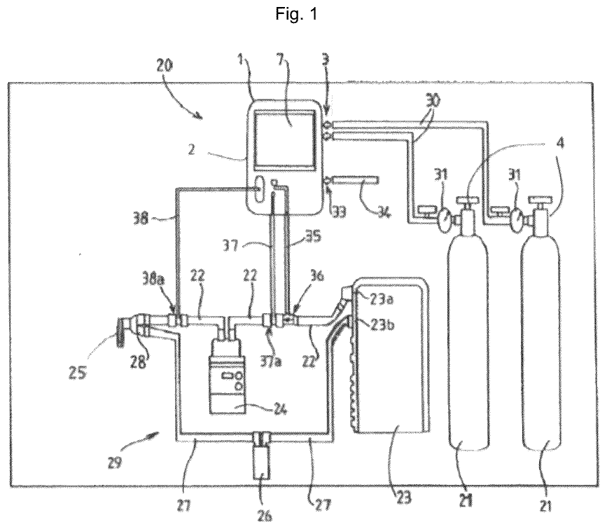

In general, the administration of iNO to a patient suffering from severe ARDS and initially presenting criteria for placing on ECMO can be carried out, in the context of the latter's treatment, by using ventilation equipment 20, that is to say gas supply equipment, such as that shown diagrammatically in FIG. 1.

In the embodiment shown, the ventilation equipment 20 comprises an NO delivery apparatus 1 cooperating with a medical ventilator 23 and a patient circuit 29, so as to inject NO into the patient circuit 29 which comprises an inhalation branch 22 and an exhalation branch 27. The inhalation branch 22 furthermore conveys a respiratory gas containing a high proportion of oxygen, typically pure oxygen or an O₂/N₂ mixture (for example containing at least 60 vol. % of O₂) supplied by a medical ventilator 23.

Two pressurized gas cylinders 21 each containing an NO/N₂ gas mixture, namely in this case an NO/N₂ gas mixture containing for example 800 ppmv of NO (remainder N₂), supply NO/N₂ mixture to the NO delivery device 1 via gas supply lines 30, such as flexible hoses or conduits or the like, which may be equipped with devices 31 for regulating and/or monitoring the gas pressure, such as gas regulators, manometers, etc. The cylinders 21 are equipped with a gas distribution valve 4.

The gas feed lines 30 are connected to one or more gas inlets 3 of the NO delivery device 1, which supply an internal gas passage serving to convey the gas within the NO delivery device 1, that is to say in the housing 2 or the outer shell of the NO delivery device 1.

The NO delivery device 1 also comprises an oxygen inlet 33 fluidically connected, via an oxygen feed line 34 such as a flexible hose or the like, to an oxygen source (not shown), for example a pressurized oxygen cylinder or a hospital network, that is to say an oxygen supply line arranged in a hospital building.

The medical ventilator 23 which supplies a respiratory gas flow containing a high proportion of oxygen, such as pure oxygen or an oxygen/nitrogen (N₂/O₂) mixture, for example containing at least 60 vol. % of O₂, and the NO delivery device 1 are in fluidic communication with the inhalation branch 22 of the patient circuit 29 so as to supply thereto the NO-based gas mixture to be conveyed the patient, this gas mixture being formed by mixing the flow from the medical ventilator 23 and the flow containing the NO, i.e. the NO/N₂ gas mixture, delivered by the NO delivery device 1. The ventilator 23 comprises regulating means, such as one or more keys or the like, making it possible in particular to set the desired FiO₂ but also to regulate the characteristics of the delivered flow, for example flow rate, pressure and/or the like.

The NO delivery device delivers or injects the NO/N₂ mixture, here at 800 ppmv of NO, into the inhalation branch 22 via an injection conduit or line 37, so as to inject (at 37a) the flow of NO/N₂ into the flow of O₂ or of oxygen/nitrogen mixture delivered by the medical ventilator 23 and conveyed by the inhalation branch 22, also called gas feed line.

The inhalation branch 22 further comprises a gas humidifier 24 arranged downstream of the site 37a where NO is injected into the inhalation branch 22 of the patient circuit 29. It makes it possible to humidify the flow of gas, e.g. NO/N₂/O₂ mixture, before it is inhaled by the patient to be treated by means of a respiratory interface, typically a tracheal intubation tube 25.

The exhalation branch 27 constitutes a line for recovering the gases exhaled by the patient. It is connected to the inhalation branch 22 via a connecting piece 28, preferably a Y-piece, thus defining the patient circuit 29.

The inhalation branch 22 is fluidly connected, upstream, to an outlet port 23a of the medical ventilator 23 so as to recover and convey the gas, typically the O₂ or an N₂/O₂ mixture, delivered by the medical ventilator 23, while the exhalation branch 27 is fluidly connected, downstream, to an inlet port 23b of the medical ventilator 23 so as to return to the medical ventilator 23 all or part of the flow of the gases exhaled by the patient, that is to say the gases rich in CO₂.

The exhalation branch 27 can comprise one or more optional components, for example a CO₂ removal device 26, i.e. a CO₂ trap, such as a hot container or the like, used to remove CO₂ from the patient's exhaled gases, or a filter or the like.

Optionally, a flow sensor 36, for example of the hot wire or pressure differential type, is arranged on the gas feed line, that is to say the inhalation branch 22, between the ventilator 23 and the humidifier 24, and is connected to the NO delivery device 1 via a flow rate measuring line 35. This arrangement measures the flow rate of gas delivered by the ventilator 23, such as O₂ or an N₂/O₂ mixture, and circulating in the inhalation branch 22, upstream of the connection site 37a of the injection conduit or line 37 where the NO/N₂/O₂ mixture is made. This makes it possible to better regulate the delivery of the NO flow by the NO delivery device 1.

A gas sampling line 38 fluidically connects the inhalation branch 22 to the NO delivery device 1. It is fluidically connected (at 38a) to the inhalation branch 22, between the humidifier 24 and the joining piece 28, i.e. the Y-piece, typically in the immediate vicinity of the joining piece 28, and also to an inlet port 102 of the NO delivery device 1, for example a port carried by a connector, coupling or the like allowing the connection of the gas sampling line 38, such as a flexible hose or the like. It allows gas samples to be taken for verification of compliance and to be conveyed to the NO delivery device 1 where they are analysed in an internal gas analyser.

In particular, it should be verified that their composition (i.e. dosage) conforms with that of the desired NO/O₂/N₂ gas mixture to be administered to the patient, in particular in order to ensure that it does not contain excessive amounts of toxic NO₂ species, that its oxygen content is not hypoxic, and that its NO content corresponds to the desired dosage. This conformity check is conventionally carried out by dedicated measuring means, typically NO₂, NO and O₂ sensors, which themselves have to be calibrated periodically, for example every week.

The NO delivery apparatus 1 comprises, in a conventional manner, a rigid housing, for example made of polymer, through which an internal gas passage (not visible) passes, such as a gas conduit or the like, in order to convey the NO/N₂ flow fed through the one or more gas feed lines 30, the latter being supplied by the NO/N₂ mixture cylinders 21. The internal gas passage fluidically connects the gas inlet(s) 3 of the NO delivery device 1 to the injection line 37 in such a way as to convey the NO-based gas flow between them.

Conventionally, valve means (not shown), i.e. one or more valve devices, for example a plurality of solenoid valves arranged in parallel or one or more proportional (solenoid) valves, are arranged on the internal gas passage in order to control the gas flow which circulates therein in the direction of the injection line 37. The valve means are controlled by control means, i.e. one or more control devices, arranged in the housing, typically an electronic card comprising one or more microprocessors, typically one or more microcontrollers, implementing one or more algorithms.

The control means make it possible in particular to regulate or control the gas flow rate by controlling the valve means, typically to open or close said valve or valves, in order to obtain a gas flow rate determined and/or calculated by the control means from a value set/fixed by the user, and as a function of the flow rate of gas, i.e. air, delivered by the ventilator 23 and measured by the flow sensor 36 which is connected to the NO delivery device 1 by the flow rate measuring line 35. The flow rate measurements of the flow delivered by the ventilator 23 and circulating in the inhalation branch 22 are supplied to the control means.

The internal gas passage can also comprise one or more flow meters (not shown) arranged upstream and/or downstream of the valve means, in order to determine the flow rate of NO-based gas circulating in the NO delivery device 1. The flow meter can be of the pressure-differential type, the hot-wire type or some other type. It cooperates with the control means in order to provide them, here again, with measurements of the flow rate of the NO/N₂ flow.

Furthermore, the NO delivery device 1 also comprises a graphical user interface (GUI) comprising a graphical display 7, preferably a touch screen, that is to say a touch panel, serving to display various information items or data, icons, curves, alerts, etc., and also virtual selection keys and/or panes or windows, in particular for making choices, selections or for entering information, such as desired values (e.g. flow rate, dose of NO, etc.), or any other information or data useful to the healthcare provider, in particular the desired dose of NO in the final NO/O₂/N₂ mixture.

The control means (not visible) comprise, for example, an electronic control card and a microprocessor-based control unit, typically a microcontroller or the like. They make it possible to adjust or control all the electromechanical elements of the NO delivery apparatus 1. More precisely, the control card preferably integrates the control unit and is configured to control and also to analyse the signals coming from the various components of the NO delivery apparatus 1, such as the pump, sensors 32, etc.

The electrical supply to the NO delivery apparatus 1, in particular to the components requiring electrical current in order to operate, such as the control means, the graphical display 7, etc., is provided conventionally by an electrical current source and/or electrical supply means (not shown), for example a connection to the mains current (110/220V), such as an electrical cord and connection socket, and/or one or more electric, preferably rechargeable, batteries and/or a current transformer.

For example, the user can select or set, via the GUI of the NO delivery apparatus 1, the NO content to be administered to the patient, for example 20 ppmv, and the control means then control the NO delivery apparatus 1 to deliver the NO/N₂ mixture at a flow rate that is suitable for obtaining the mixture NO/N₂ and air (or O₂/N₂) at the correct concentration. This is done in particular by virtue of the values coming from the flow sensor 32 which measures the flow rate (or pressures) of the air (or O₂/N₂) flow coming from the ventilator 23.

Typically, the final mixture supplied to the patient, which contains NO at the desired concentration, oxygen and nitrogen, generally contains less than 40 ppmv of NO, for example about 10 to 20 ppmv approximately.

In general, the present invention is particularly well suited to the treatment of severe ARDS patients presenting ECMO criteria, regardless of the aetiology of ARDS, who are under artificial ventilation and intubated, i.e. under invasive ventilation, typically patients in intensive care or the like. Advantageously, the iNO is administered rapidly after intubation of the patient, preferably within 24 hours after intubation.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

What is claimed is:

1. A method for treating a patient suffering from acute respiratory distress syndrome (ARDS), comprising:
a) providing or selecting an adult patient suffering from a severe ARDS characterized by:
(i) a severity index such that: PaO₂/FiO₂≤80 mmHg or
(ii) a carbon dioxide partial pressure (PaCO₂) and a blood pH such that PaCO₂≥60 mm Hg and pH<7.25,
b) administrating by inhalation a gaseous drug containing nitric oxide (NO) to said patient,
wherein PaO₂, PaCO₂ and/or PH are measured in the patient prior to administration of the gaseous drug and FiO₂ is an amount of oxygen supplied to the patient.

2. The method according to claim 1, characterized in that the gaseous drug comprises NO and nitrogen.

3. The method according to claim 2, characterized in that the gaseous drug containing NO and nitrogen (N₂) is mixed with an oxygen-containing gas, prior to administration to the patient.

4. The method according to claim 3, characterized in that the NO/N₂ gaseous drug is diluted with an oxygen-containing gas containing at least 20 vol. % oxygen, prior to administration to the patient, thereby obtaining a final mixture containing oxygen, nitrogen and less than 40 ppmv of NO as a dose of NO that is subsequently administered to the patient.

5. The method according to claim 3, characterized in that the oxygen-containing gas is oxygen or an oxygen-nitrogen mixture.

6. The method according to claim 4, characterized in that the gaseous drug mixed with the oxygen-containing gas, prior to administration to the patient, contains from 200 to 2000 ppmv of NO and N₂ for the rest, preferably from 200 to 1000 ppmv of NO.

7. The method according to claim 1, characterized in that the patient is an adult of at least 40 years old.

8. The method according to claim 1, characterized in that the patient is suffering from a severe ARDS characterized by a severity index such that:
a. 30 mHg≤PaO₂/FiO₂≤80 mmHg.

9. The method according to claim 1, characterized in that the patient is suffering from a severe ARDS characterized by a severity index such that:
a. 40 mmHg≤PaO₂/FiO₂≤80 mmHg.

10. The method according to claim 1, characterized in that the patient has one or more comorbidities selected from hypertension, diabetes, immunodeficiency and chronic obstructive pulmonary disease (COPD).

11. The method according to claim 1, characterized in that it is administered by inhalation for 24 hours to 96 hours.

12. The method according to claim 1, characterized in that it is administered by inhalation through a tracheal intubation tube.

13. The method according to claim 1, characterized in that the patient is an adult presenting with refractory hypoxaemia prior to treatment with NO.

14. The method according to claim 1, characterized in that the gaseous drug containing nitric oxide (NO) administrated to said patient in step b) is a gaseous mixture containing at least 20% vol. of oxygen, nitrogen and less than 40 ppmv of NO, preferably from 5 to 30 ppmv of NO.
